(19) United States
(12) Patent Application Publication
(10) Pub. No.: US 2006/0079931 A1
(43) Pub. Date: Apr. 13, 2006

(54) Surgical deep needle driver

(76) Inventors: Paul J. Brennan, Rochester, NY (US); Amit Kumar, Cleveland Heights, OH (US)

Correspondence Address: BASCH & NICKERSON LLP, 1777 PENFIELD ROAD, PENFIELD, NY, 14526, US

(21) Appl. No.: 11/186,385

(22) Filed: Jul. 21, 2005

Related U.S. Application Data
(60) Provisional application No. 60/589,579, filed on Jul. 21, 2004.

(51) Int. Cl. A61B 17/28

(52) U.S. Cl. 606/205

(57) ABSTRACT

The disclosure is directed to an improved deep needle driver for use when working on patients having significant tissue thicknesses, where conventional instruments are too short or do not provide suitable maneuverability within deep tissue. The driver includes a pair arms bent at two positions, where the arms are pivotally joined to form a movable jaw.

Priority is claimed from U.S. Provisional Application 60/589,579 for a DEEP DRIVER, by A. Kumar and P. Brennan, filed Jul. 21, 2004, which is hereby incorporated by reference in its entirety.

### BACKGROUND AND SUMMARY

The present disclosure relates to improvements in instruments used in holding needles or suturing tissue during surgery. More particularly, the disclosure relates to an improved deep needle driver for use when working on patients having significant tissue thicknesses, where conventional instruments are too short or do not provide suitable maneuverability within deep tissue cavities.

Forceps and similar needle holders or drivers, such as depicted in FIG. 8 are well-known medical instruments. For example, the Mayo Hegar Needle Holder (e.g., P/N KM41-270, KM41-302, etc.) include jaws 10, a box lock 12, shanks 14, finger rings 18 and a ratchet 16 for holding the jaws in a generally closed position (with a needle held therebetween). The jaws of such drivers can be of milled stainless steel or may include a tungsten carbide surface for greater durability and longevity (needed to hold needles during repeated use). Such instruments are generally similar to hemostats, but are generally of a more rigid design and shorter in length. It will be apparent that shorter needle drivers may be used for working close to a surface whereas longer drivers are for deeper cavities.

As those familiar with surgical procedures will appreciate, although longer needle drivers may be used to reach into deeper cavities, the inherent problem with such use is the angle at which the driver must be held in order to reach into the cavities. This problem is further exacerbated by surgery on patients having significant fatty tissue deposits, which in some cases, make the use of conventional, straight drivers difficult, if not impossible. In order to permit the surgeon to maintain a typical or preferred orientation of his/her hands when suturing or manipulating other materials within a deep cavity, the following disclosure characterizes improvements to a deep driver or similar instrument.

Disclosed in embodiments herein is a surgical instrument, comprising: a pair of arms, said arms each including an opposing surface on a first end and a feature for contact with a user's hand on a second end, wherein said arms are formed so as to have at least two bends therealong defining a midsection and uppermost section, with the first bend proximate the opposing surface and thereby separating the opposing surface from the midsection; and a pivot proximate the first bend of said arms, said pivot operatively connecting said arms and forming a joint, wherein movement of the second end of said arms causes the opposing surfaces on the first ends to move relative to one another.

Also disclosed in embodiments herein is a needle driver, comprising two arms and a hinge joining the two arms together, each of the two arms having a first end for grasping a needle, a second end for actuation by a user's fingers, a first bend near the first and, and a second bend near the second end in a direction opposite to a direction of the first bend.

### BRIEF DESCRIPTION OF THE DRAWINGS

FIGS. 1 and 2 are exemplary representations of needle drivers as disclosed;

FIGS. 3A-3D are exemplary representations of an alternative embodiment of a needle driver, respectively including the assembled and separated components;

FIG. 4 is an illustration of the driver in an open configuration;

FIGS. 5A and 5B illustrate a locking mechanism in one embodiment of the driver;

FIGS. 6A, 6B and 7 illustrate an alternative embodiment of the needle driver in orthographic and perspective views; and

FIG. 8 is an illustrative representation of a prior art needle driver.

The following description will be set forth in connection with a preferred embodiment, however, it will be understood that there is no intent to limit the claimed invention to the embodiments described. On the contrary, the intent is to cover all alternatives, modifications, and equivalents as may be included within the spirit and scope of the appended claims.

### DETAILED DESCRIPTION

For a general understanding of the disclosed embodiments, reference is made to the drawings. In the drawings, like reference numerals have been used throughout to designate identical elements. In describing the embodiments, the following term(s) have been used in the description. The term “driver” or “needle driver” is intended to encompass not only those surgical instruments that are intended for the grasping and manipulation of suture needles, but to similarly constructed instruments suitable for grasping other surgical implements, body tissue and the like.

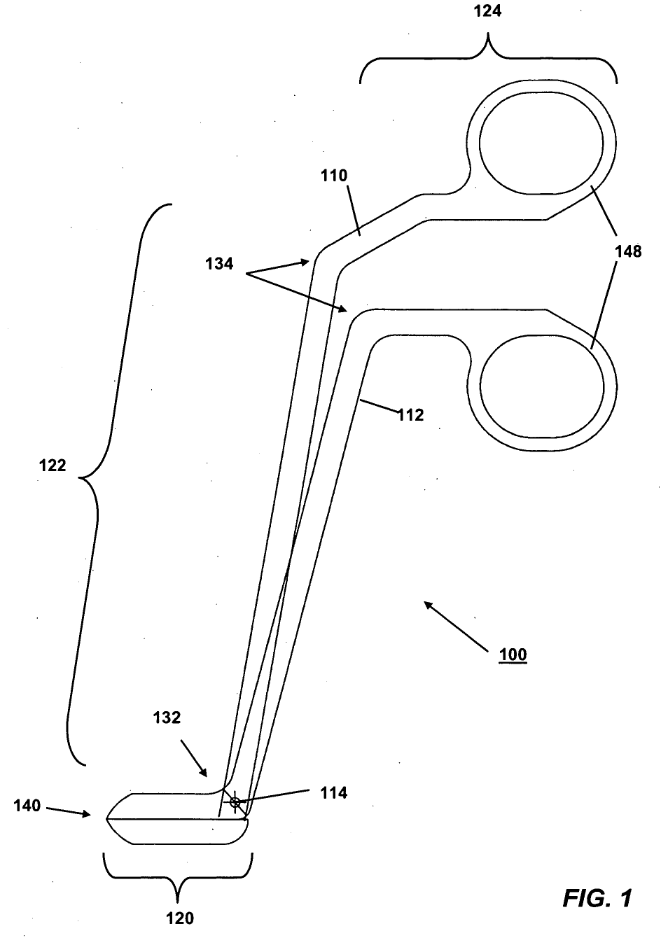

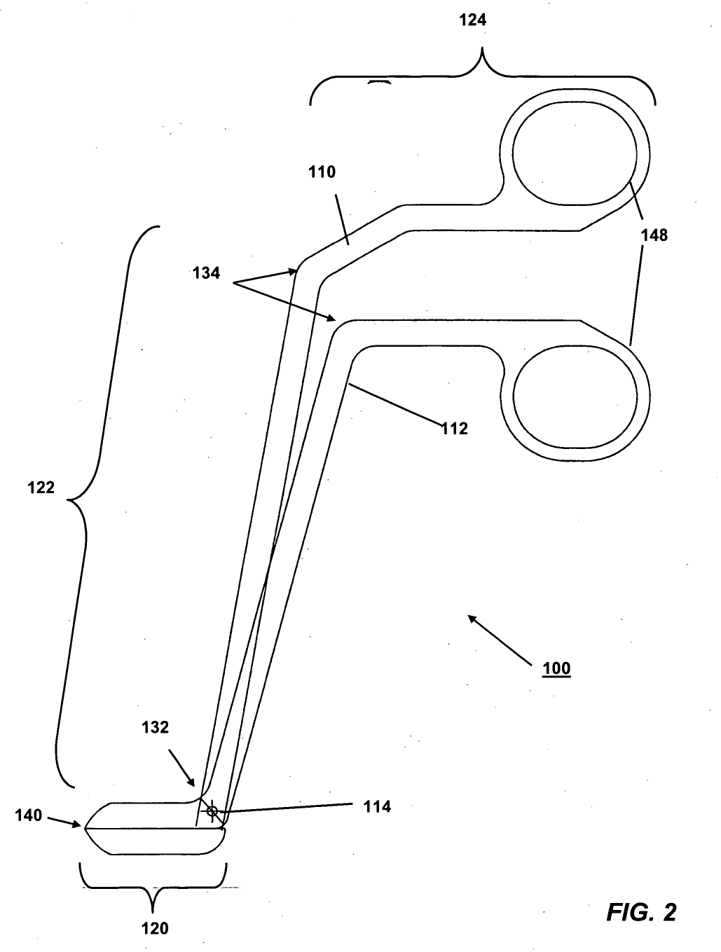

Referring now to FIGS. 1 and 2, there are depicted two alternative deep needle drivers that overcome the aforementioned problems when dealing with sutures and the like in a deep cavity. It will be appreciated that such instruments would find practical application in surgical procedures conducted on obese patients, where the depth of a cavity is further exacerbated by fatty tissue, making the use of conventional needle drivers difficult. Depicted in FIGS. 1 and 2 is a surgical instrument such as a needle driver 100. In its simplest form, the instrument comprises a pair of arms 110 and 112, which are hinged or pivotally connected to one another at a joint 114. Each of the arms 110 and 112, are formed of a similar shape, although it will be apparent from the various configurations set forth in the drawings, that the shapes of the two arms are somewhat different so that the arms do not interfere with one another during use of the instrument.

Continuing with FIGS. 1 and 2, the arms are divided into three sections (opposing surface section 120, middle section or midsection 122, and upper section 124) by at least two bends 130 and 134. At the lower or first end of each arm, there are opposing surfaces that form a jaw 140 therebetween. Although not depicted in particular detail, it will be appreciated that the jaw, and the associated opposing surfaces, may be of various shapes and configurations. Although the embodiments depicted in FIGS. 1 and 2, illustrate planar opposing surfaces, other shapes are contemplated and included within the scope of this disclosure. It will be appreciated that for the purposes of holding a suture needle, the surfaces should be mating surfaces (meaning a structure on one surface should not interfere with an opposing structure to prevent the jaws from closing about a needle). In alternative embodiments, the structure may be particularly designed for grasping needles or similar implements, and may include a milled or roughened surface and/or alternative surface coatings (e.g., tungsten carbide for wear resistance, etc.). Moreover, although the shape of the jaw is disclosed as a generally block-type shape for purposes of illustration, it will be appreciated that the dimensions of the jaw and mating surfaces may be modified, and that various rounding and curvature of the tips and outer surfaces may be employed to make the instrument suitable for use in surgical procedures. In yet another embodiment, jaw 140 may be designed such that when the opposing surfaces come into contact, there is a slight groove or opening between the surfaces into which a needle or the like may be more easily inserted and held in a clamped manner by the user when the jaws are closed.

On the opposite end of each arm 110 and 112, is a finger/thumb ring or a similar feature 148 for contact with a user's hand. Although both arms are depicted with finger/thumb rings, it is also possible that one or both arms terminate at the upper end with alternative features such as a knurled grip to facilitate the manipulation of the driver with other than a user's finger and thumb. Such a configuration may provide greater strength and less fatigue when working with elongated drivers in deep surgical cavities.

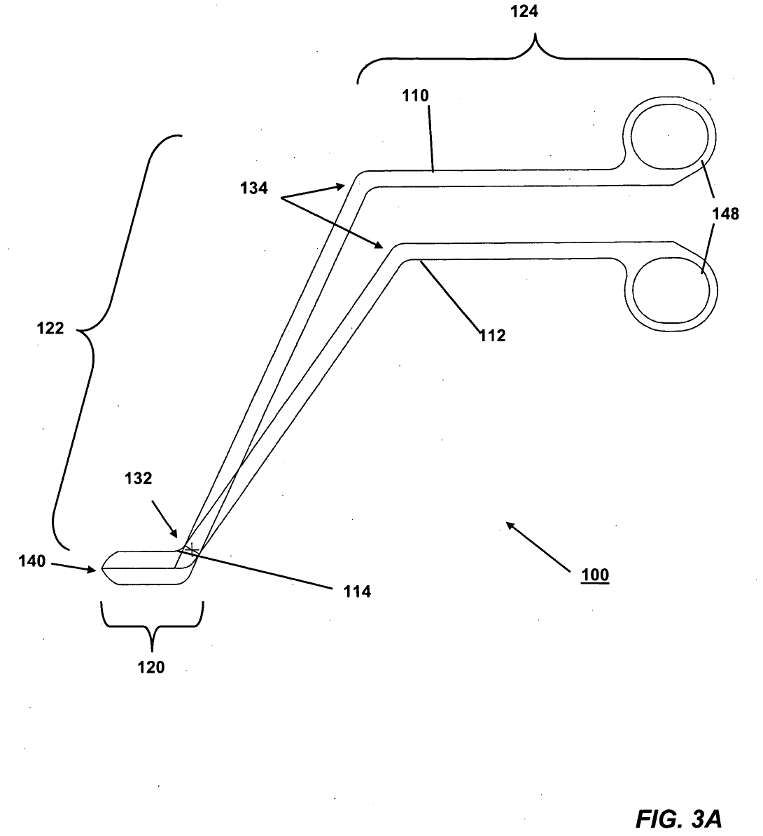

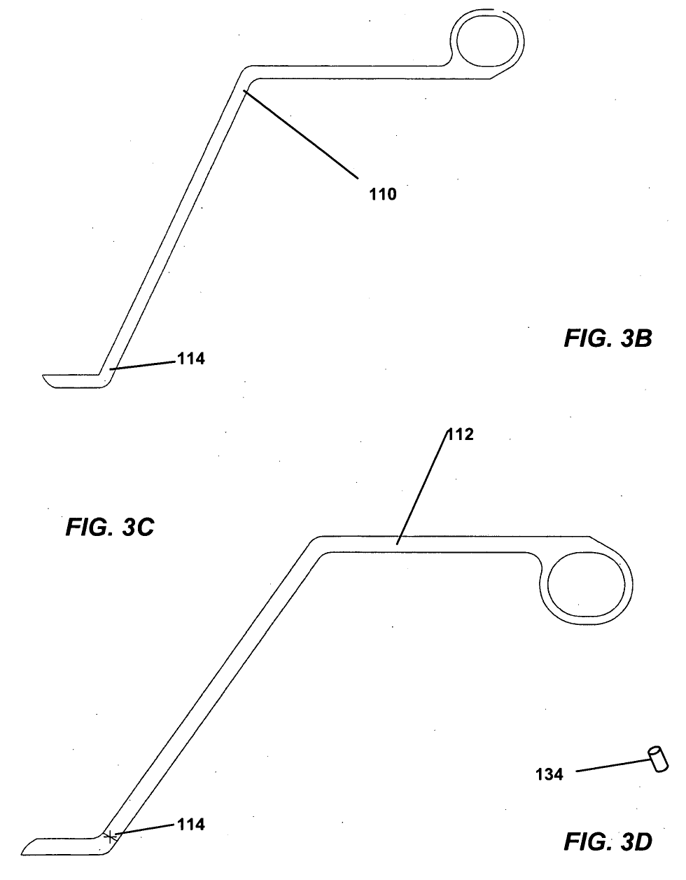

As described above, each arm is formed to have at least two bends (132, 134) therealong, which serve to define the midsection 122 and uppermost section 124, where the first bend is generally proximate the opposing surfaces. A pivot 114 is also located proximate the first bend and the opposing surfaces of the arms 110 and 112. Although generally depicted as a pin-type pivot as shown in FIG. 3D, it will be appreciated that various alternative hinge or pivot mechanisms may be employed in accordance with the driver 100. For example, in one embodiment, the pivot may be accomplished using a joint, such as a lap joint or a box joint operatively connecting the arms. Thus, when joined at the pivot 114, movement of the other ends of the arms causes the opposing surfaces to move relative to one another. It is further contemplated that the joint may include a washer or similar device located at the pivot, to reduce or prevent friction between the arms 110 and 112.

As further illustrated in FIGS. 1 and 2, the two bends of arm 112 are of approximately equal angles, but of opposite direction. This assures that the uppermost section of the arm is generally parallel to the opposing surface, making the surgical instrument suitable for use in a manner typical of conventional drivers, yet appropriate, due to the elongated midsection, for use in deep tissue or body cavities.

As will be appreciated, angle 134 of the upper arm may be accomplished with a several nearby bends, thus preventing the two arms of the driver from interfering with one another when the upper sections are grasped and squeezed together by the user. In other words, in at least one arm the second bend may include a pair of minor bends to assure that the upper sections of the arms do not interfere with one another when a user urges them together. The sum of the angle of the minor bends will approximate the angle of the first bend in the arm, thereby assuring that the uppermost section 124 is generally parallel with the lower or opposing surface section 120.

In the various design alternatives set forth in FIGS. 1 through 7, it will be apparent that different sizes of the driver may be constructed. The size may depend upon the particular application, particularly having longer midsections for use in deeper cavities. In the various embodiments, the range of midsection lengths is about 2 inches to about 10 inches, and typically would be in the range of about 4 to 6 inches. It will, however, be appreciated that particular situations may require longer or shorter lengths than those set forth. In the embodiments of FIGS. 1 and 2, the length of the midsection 122 is generally greater than the length of the upper section 124, although this not a requirement.

Referring also to FIGS. 3A-3D, the embodiment depicted therein is an alternative having elongated upper sections 124 for both arms 110 and 112, where the upper section lengths are several inches and are approximately the length of the midsection. It is believed that such a configuration may provide more gripping force at jaw 140 for the same equivalent force applied to the embodiments of FIGS. 1 and 2. FIGS. 3B and 3C respectively illustrate the detailed shape of arms 110 and 112. Moreover, using one of the joint configurations noted above, arms 110 and 112 may be attached using a pin 134. Although it is believed that a permanent pin or equivalent pivot mechanism (e.g., rivet) may be preferable, alternative pivot mechanism such as removable screws, bolts and the like are also contemplated. The selection of the pivot mechanism may be determined not only for purposes of manufacturability, but also from the perspective of cleaning and sterilization of the device. For the latter reason, it is also contemplated that the driver 100 be manufactured from stainless steel, or a formable polymer suitable for withstanding conventional sterilization chemicals and treatments.

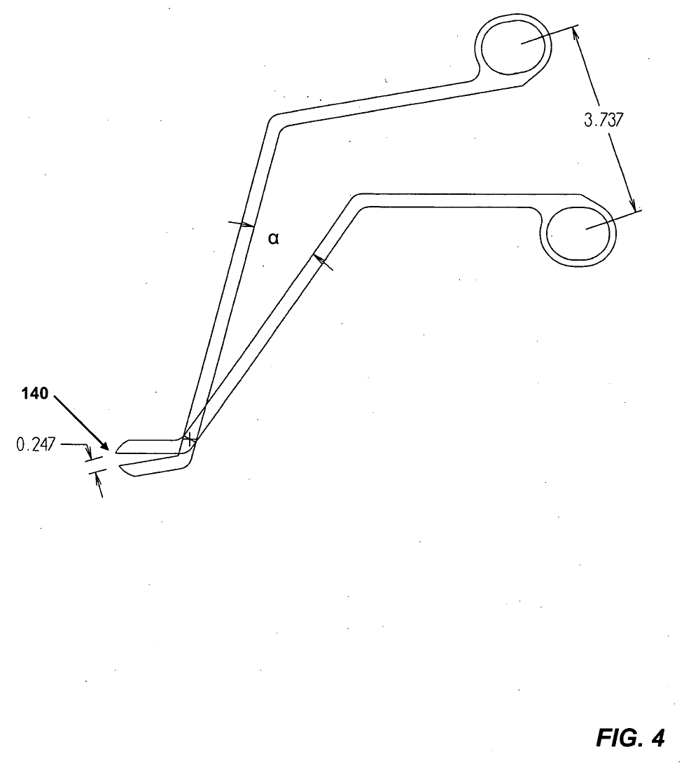

Referring also to FIG. 4, there is shown the driver of FIG. 3A, in an opened configuration. It will be appreciated that as angle alpha (a) changes from a closed angle of approximately 10-degrees to an opened angle of approximately 20-degrees (as shown), the jaws 140 are opened to receive a suture needle or the like therein. In one embodiment, the jaws may have an opening at the outermost end of approximately 0.247 inches in response to a user opening the uppermost section by approximately 3.737 inches. Again, it will be appreciated that different dimensional configurations will alter the relationship between the jaws 140 and the uppermost sections of the arms 110 and 112.

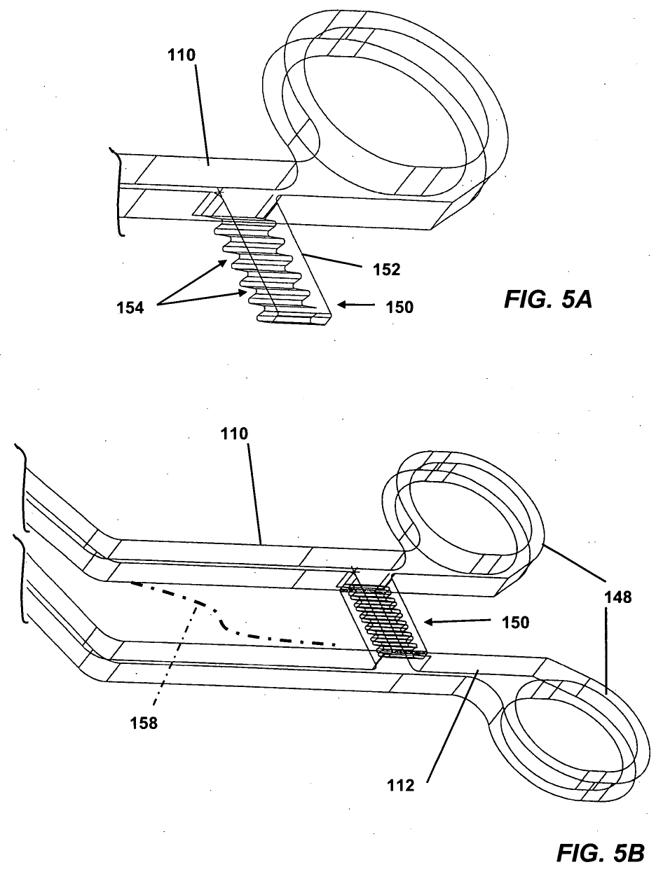

Turning next to FIGS. 5A and 5B, there is depicted a further aspect of the driver in accordance with an alternative embodiment. In particular, the upper sections of each arm have associated therewith a locking mechanism 150. As depicted in FIGS. 5A and 5B, mechanism 150 is a ratchet mechanism, wherein each arm includes a tab 152 extending therefrom, where at least one side has a series of angled grooves or cuts 154, and the grooves may be suitably mated with grooves on a tab of the opposing arm to lock the arms. Such mechanisms are well known to those familiar with surgical instruments. Although a ratchet is shown in the figures, it will be appreciated that other mechanism may be employed to bias, hold or lock the driver 100 in a generally closed position—where the opposing surfaces at the opposite ends of the arms are urged to contact one another (e.g., to hold a suture needle therebetween). Alternatives considered include single springs 158, double springs, springs with rollers and a double spring with a ball and socket joint. Again, such mechanisms are known for use in medical instruments as well as other hand-operated tools.

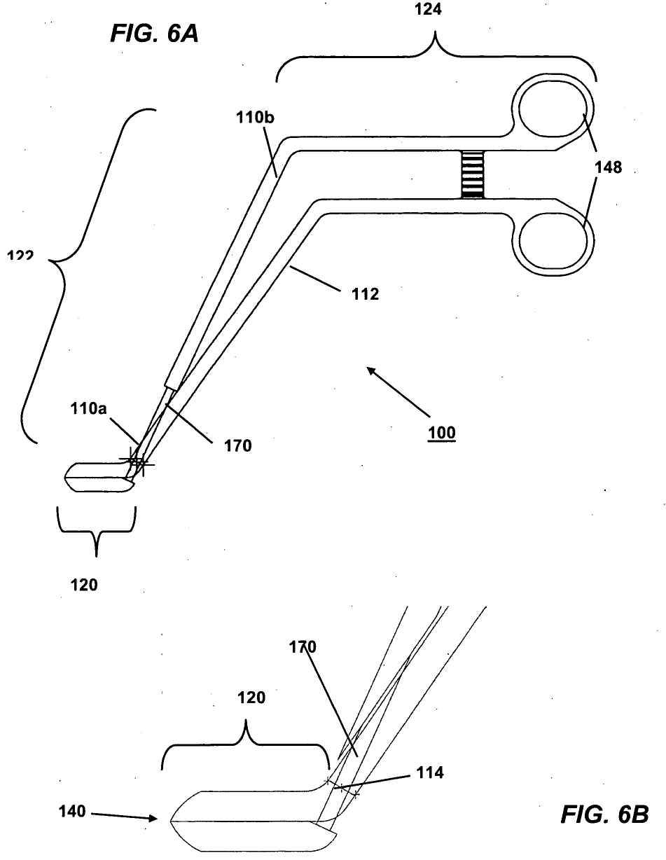

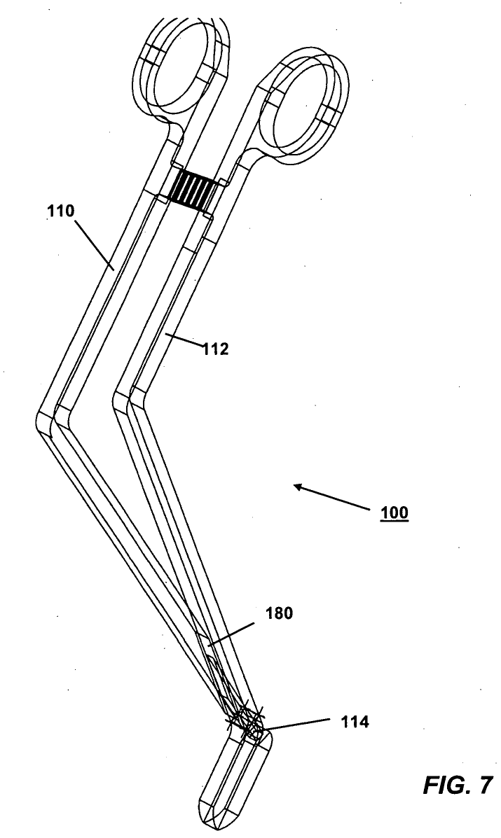

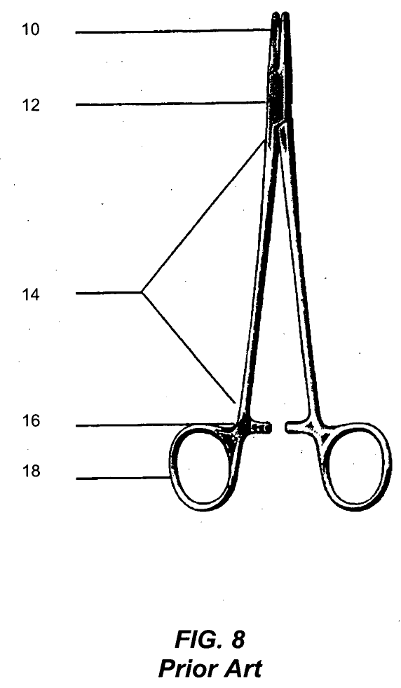

Referring now to FIGS. 6A, 6B and 7, depicted therein is yet another alternative embodiment that is believed particularly suitable for ease of manufacturing and use. In particular, the driver 100 is formed with an arm 110 that is made from at least two components 110a and 110b. The arm 110 is divided in the midsection, and lower component 110a is joined or mated with upper component 110b to complete the arm 110. As will be appreciated from FIG. 6B, component 110a includes an opposing surface section 120 as previously described, but includes a reduced size elongated section 170, where elongated section 170 is of a dimension smaller than that of component 110b and of arm 112. This facilitates the assembly of arm 110 after the elongated section 170 has been inserted through an aperture 180 in arm 112. In this design, a completely boxed hinge or pivot 114 is provided, and the need for a second angle at the upper section of arm 110 is eliminated—the aperture 180 providing clearance to elongated section 170 over a full range of motion for the driver.

It is further contemplated, based upon the configuration of FIGS. 6A-7, that a driver 100 may be produced with varying lengths of midsections 122, or that a midsection component of one length may be substituted for a midsection component of a different length, on both arms of course, so as to provide an adjustable driver. Such an instrument would be of benefit in situations where a user may wish to have an instrument suitable for reaching into deeper cavities, but does not wish to purchase several drivers of different sizes. The design of FIGS. 6A-7, in addition to the features noted above, permits the substitution of different length mid-sections at the user's discretion.

It will be appreciated that various of the above-disclosed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims.

What is claimed is:

1. A surgical instrument, comprising:
a pair of arms, said arms each including an opposing surface on a first end and a feature for contact with a user's hand on a second end, wherein said arms are formed so as to have at least two bends therealong defining a midsection and uppermost section, with the first bend proximate the opposing surface and thereby separating the opposing surface from the midsection; and
a pivot proximate the first bend of said arms, said pivot operatively connecting said arms and forming a joint, wherein movement of the second end of said arms causes the opposing surfaces on the first ends to move relative to one another.

2. The surgical instrument of claim 1, wherein the second bend is in a direction opposite the first bend.

3. The surgical instrument of claim 2, wherein in at least one arm, the first and second bend are each of equal but opposite angles so that the uppermost section of the arm is generally parallel to the opposing surface.

4. The surgical instrument of claim 3, wherein in at least one arm, the second bend comprises a pair of minor bends so as to assure that the upper sections of the arms do not interfere with one another when a user urges them together.

5. The surgical instrument of claim 1 further including a washer located at the pivot, between the arms.

6. The surgical instrument of claim 1, wherein a length of said midsection is in the range of about 2 inches to about 10 inches.

7. The surgical instrument of claim 6 wherein the length of the midsection is greater than the length of the uppermost section.

8. The surgical instrument of claim 1 wherein the feature for contact with a user's hand includes a ring through which a user's digit may be inserted.

9. The surgical instrument of claim 1 further comprising, at the uppermost section of each arm, a locking mechanism to hold the arms in a generally closed position where said opposing surfaces are urged to contact one another.

10. The surgical instrument of claim 9, wherein said locking mechanism comprises a ratchet lock.

11. The surgical instrument of claim 9, wherein said locking mechanism comprises a spring.

12. The surgical instrument of claim 1 wherein said joint is a lap joint.

13. The surgical instrument of claim 1 wherein said joint is a box joint.

14. The surgical instrument of claim 1 wherein said joint is formed by passing a portion of one arm through an aperture in another arm and placing a pivot pin therethrough.

15. The surgical instrument of claim 14 wherein said one arm comprises at least two pieces which are operatively connected to one another during assembly of the instrument.

16. The surgical instrument of claim 1, wherein said midsection of each arm is adjustable in length.

17. A needle driver, comprising two arms and a hinge joining the two arms together, each of the two arms having a first end for grasping a needle, a second end for actuation by a user's fingers, a first bend near the first and, and a second bend near the second end in a direction opposite to a direction of the first bend.

18. The needle driver of claim 17, wherein the hinge is located at the first bend of each of the arms.

19. The needle driver of claim 17, wherein the hinge is located between the first bend and the first end of each of the arms.
